(19) United States
(12) Patent Application Publication
(10) Pub. No.: US 2017/0124709 A1
(43) Pub. Date: May 4, 2017

(54) SYSTEMS AND METHODS FOR MEDICAL IMAGE SEGMENTATION AND ANALYSIS

(71) Applicant: Massachusetts Institute of Technology, Cambridge, MA (US)

(72) Inventors: Rahul Rithe, Cambridge, MA (US); Anantha P. Chandrakasan, Belmont, MA (US)

(21) Appl. No.: 15/311,126

(22) PCT Filed: May 14, 2015
(86) PCT No.: PCT/US15/30898

§ 371 Date: Nov. 14, 2016

Related U.S. Application Data
(60) Provisional application No. 61/996,818, filed on May 14, 2014.

(51) Int. Cl. G06T 7/00; G06T 5/40; G06K 9/46; G06K 9/62; A61B 5/00; H04N 5/232; H04N 5/225; G06T 7/11; G06K 9/20; G06T 7/90; G06K 9/22

(52) U.S. Cl. CPC G06T 7/0016; G06T 7/90; G06T 5/40; G06K 9/4604; G06K 9/4652; G06K 9/6211; G06K 9/6298; G06K 9/4671; G06K 9/228; H04N 5/23293; H04N 5/2256; G06T 7/11; G06K 9/2036; G06K 9/2018; A61B 5/0077; A61B 5/4848; A61B 5/742; A61B 5/444; A61B 5/0075; G06T 2207/30096; G06T 2207/10024; G06T 2207/20161; G06T 2207/30088; G06T 2207/10004; H04B 5/0025

(57) ABSTRACT

The present disclosure includes systems, methods, and computer-readable medium for monitoring and analyzing skin lesions. A sequence of images are be received, and color correction, contour detection, and feature detection are performed on the images. A progression factor is determined based on a comparison of the an area of the lesion between images. A system for monitoring a progression of a skin lesion is provided that includes a portable imaging device to aid in capturing images of the lesion, and a user device configured to analyze the images and determine a progression factor of the skin lesion.

### RELATED APPLICATIONS

This application claims priority to U.S. Provisional Patent Application No. 61/996,818 filed on May 14, 2014, the entire contents of the above application being incorporated herein by reference.

### BACKGROUND

Chronic skin conditions are often easily visible and can be characterized by multiple features including pigmentation, erythema, scale or other secondary features. Due to its appearance on visible areas of the skin, such conditions can have a significant negative impact on the quality of life in affected children and adults.

Several surgical and non-surgical treatments are available; however, reliable objective outcome measures are currently lacking. Treatment of skin conditions aims to arrest disease progression and induce repigmentation of affected skin. In standard clinical practice, the degree of repigmentation is assessed subjectively by the physician by comparing the extent of skin lesions before and after treatment, often based on a series of clinical photographs. A variety of scoring systems are used to evaluate the treatment outcome in terms of repigmentation, making cross-study comparisons difficult. Current outcome measures are limited and include the Physician's Global Assessment (PGA), grading patients' improvement based on broad categories of percentage repigmentation over time. The Vitiligo Area and Severity Index (VASI) is another outcome metric that measures percentage repigmentation graded over area of involvement summed over body sites involved. Due to its complexity, it cannot easily be incorporated into clinical practice. Moreover, these outcome measures rely on subjective clinical assessment through visual observation, which cannot exclude inter-observer bias and can therefore have limited accuracy, reproducibility and quantifiability.

A number of conventional imaging systems have been used in medical imaging for capturing images of skin lesions for analysis. However, widespread use of these systems has been limited by factors such as size, weight, cost and complex user interface. Some of the commercially available systems are useful for eliminating glare and shadows from the field of view but do not discriminate from ambient lighting. More complex systems based on confocal microscopy, for example, trade-off portability and cost for high resolution and depth information.

### SUMMARY

Preferred embodiments of the invention relate to systems and methods for measuring body conditions and the diagnosis thereof. Preferred embodiments can include methods for image enhancement and segmentation that can be used to accurately determine lesion contours in an image and a registration method using feature matching can be used to process the images for diagnosis, such as by alignment by a sequence of images for a lesion, for example. A progression metric can be used to accurately quantify pigmentation of skin lesions. The system can include an imaging detector connected to a data processor that processes image data.

Some embodiments include a computer-implemented method for analyzing a body feature or condition such as a lesion in an image. The method includes receiving a first image of the lesion, performing color correction on the first image such as by performing histogram equalization on a color channel, performing contour detection on the first image, performing feature detection on the first image, and storing results of image correction. The method can also include receiving a sequence of images wherein the images correspond to the lesion represented in the first image, and the sequence of images represent the lesion over a period of time. The method further includes performing color correction on the sequence of images, performing contour detection on the sequence of images, performing feature detection on the sequence of images, and determining a progression factor based on a comparison of an area of the lesion in the first image and of the lesion in the sequence of images. In the method, the color channel comprises a red color channel, a green color channel, and a blue color channel, and the histogram is performed on each of the color channels independently. In the method, performing the contour detection comprises performing a level set method using a region-based image segmentation scheme. Further in the method, performing the feature detection includes performing a Scale Invariant Feature Transform (SIFT) feature matching.

Another embodiment includes a method for monitoring a progression of a lesion via images. The method includes receiving a sequence of images wherein a first image of the sequence of images is indicated, performing color correction on the sequence of images including performing histogram equalization on a color channel, performing contour detection on the sequence of images, performing feature detection on the sequence of images, and determining a progression factor based on a comparison of an area of the lesion in the first image and of the lesion in the sequence of images. In the method, the sequence of images are captured by a user using a mobile device. The method further includes sending the sequence of images and the progression factor to another user device, receiving diagnosis and treatment information from the other user device, and displaying the diagnosis and treatment information on the device. In the method, the sequence of images is encrypted on the user device, and the encrypted sequence of images are sent to the other user device.

Yet another embodiment includes a system for monitoring a progression of a skin lesion. The system includes a portable imaging module configured to couple to a camera on a user device and to provide lighting to capture images of skin lesions, and the user device comprising a processor-implemented module configured to analyze images of skin lesions and determine a progression factor of the skin lesion based on a change in the area of the skin lesions. The user device is further configured to perform color correction on the images, perform contour detection on the images, perform feature detection on the images, and determine a progression factor based on a comparison of an area of the lesion between the images. In the system, the color channel comprises a red color channel, a green color channel, and a blue color channel, and the histogram is performed on each of the color channels independently. In the system, performing the contour detection comprises performing a level set method using a region-based image segmentation scheme. Further in the system, performing the feature detection includes performing SIFT feature matching.

Another embodiment includes a non-transitory computer readable medium storing instructions executable by a processing device, where execution of the instructions causes the processing device to implement a method for monitoring a progression of a lesion via images. The instructions include receiving a sequence of images wherein a first image of the sequence of images is indicated, performing color correction on the sequence of images including performing histogram equalization on a color channel, performing contour detection on the sequence of images, performing feature detection on the sequence of images, and determining a progression factor based on a comparison of an area of the lesion in the first image and of the lesion in the sequence of images. The sequence of images are captured by a user using a mobile device having an imaging sensor such as a CMOS imaging device. The mobile device can comprise a handheld camera having a wired or wireless networking communication device to enable a connection that transfers image data and medical records data to a remote server for further diagnostic use, display and storage. The handheld camera device can comprise a hand-carried mobile telephone having integrated display, processing and data communication components. This enables patients to use their personnel communicating devices to record and transmit images for processing in accordance with preferred embodiments of the invention. The stored instructions further include sending the sequence of images and the progression factor to another user device, receiving diagnosis and treatment information from the other user device, and displaying the diagnosis and treatment information on the device. The sequence of images is encrypted on the user device, and the encrypted sequence of images are sent to the other user device. The mobile device can include a first light source or is adapted to connect to a detachable light source. The detachable, or second light source, can be a white light source and/or a multispectral light source that emits light at selected wavelengths or wavelength bands.

### BRIEF DESCRIPTIONS OF THE DRAWINGS

Some embodiments are illustrated by way of example in the accompanying drawings and should not be considered as a limitation of the invention:

FIG. 1 is a flowchart of an example overall process flow for analyzing skin images for lesions, according to a preferred embodiment;

FIGS. 2A-2F show images of two different skin lesions where the color correction by histogram matching process has been performed on the images, according to a preferred embodiment;

FIGS. 3A-3C show the evolution of the contours for two different skin lesions, according to a preferred embodiment;

FIGS. 4A-4B shows sequences of images with their R, G, B histograms and the outputs after color correction, according to a preferred embodiment;

FIG. 5 shows a sequence of image segmentations using level set method for lesion contour detection, according to a preferred embodiment;

FIG. 6 shows a pair of images of the same lesion with some of the matching features identified on them, according to a preferred embodiment;

FIG. 7 shows a sequence of image registrations based on matching features with respect to a reference image at the beginning of treatment, according to a preferred embodiment;

FIGS. 8A-8B show images where contour detection is performed and then the images are then aligned by feature matching, according to a preferred embodiment;

FIGS. 9A-9C show sequences of images generated for a lesion with known change in area and the analysis of the sequence of images, according to a preferred embodiment;

FIG. 10 is a diagram of an example portable imaging module with multispectral polarized light for medical imaging, according to a preferred embodiment;

FIG. 11 is a side view and angle view of a portable imaging module mounted on a mobile device, according to a preferred embodiment;

FIG. 12 is a block diagram illustrating a mobile device for implementing systems and methods associated with a retinopathy workflow, evaluation and grading application, according to a preferred embodiment;

FIG. 13 shows an example graphical user interface for analysis and monitoring of skin lesions, according to a preferred embodiment;

FIG. 14 is a block diagram showing the imaging modules for skin lesion image analysis, according to a preferred embodiment;

FIG. 15 is a schematic of a cloud-based secure storage system for securely analyzing and transferring images and patient data, according to a preferred embodiment;

FIG. 16 is a schematic of a cloud-based processing platform for securely analyzing and transferring images and patient data, according to a preferred embodiment;

FIG. 17 illustrates a network diagram depicting a system for retinopathy workflow, evaluation, and grading for mobile devices, according to a preferred embodiment; and

FIG. 18 is a block diagram of an exemplary computing device that may be used to implement preferred embodiments of the retinopathy application described herein.

### DETAILED DESCRIPTION OF PREFERRED EMBODIMENTS

Medical imaging techniques are important tools in diagnosis and treatment of various skin conditions, including skin cancers such as melanoma. Defining the border of skin lesions and detecting their features are critical for dermatology. The importance of such measures is to allow for comparison of studies and to accurately assess changes over time. Several tissue lesions can be identified based on measurable features extracted from a lesion, making accurate quantification of tissue lesion features for clinical practice and monitoring. Prior methods suffer from a lack of repeatability. For example, the Image J software available from the National Institutes for Health have utilized a manual tracing of a lesion or feature. This approach is not reproducible and makes quantitative analysis unreliable.

An objective measurement tool for repigmentation can overcome the limitations of conventional subjective observational methods, and serve as a diagnostic tool for dermatologists. Computer vision algorithms can be applied to identify the skin lesions and extract their features, which allows for more accurate determination of disease progression. The ability to objectively quantify change over time can significantly improve a physician's ability to perform clinical trials and determine the efficacy of therapies. An example embodiment relates to image enhancement by R, G, B histogram matching and segmentation using a level set method to accurately determine the lesion boundaries in an image. Another example embodiment includes systems and methods for medical imaging for various skin conditions and monitoring the progress over time based on a Scale Invariant Feature Transform (SIFT) matching and a progress metric to quantitatively determine lesion progression over time. The progress metric may be referred to as “a fill factor” herein.

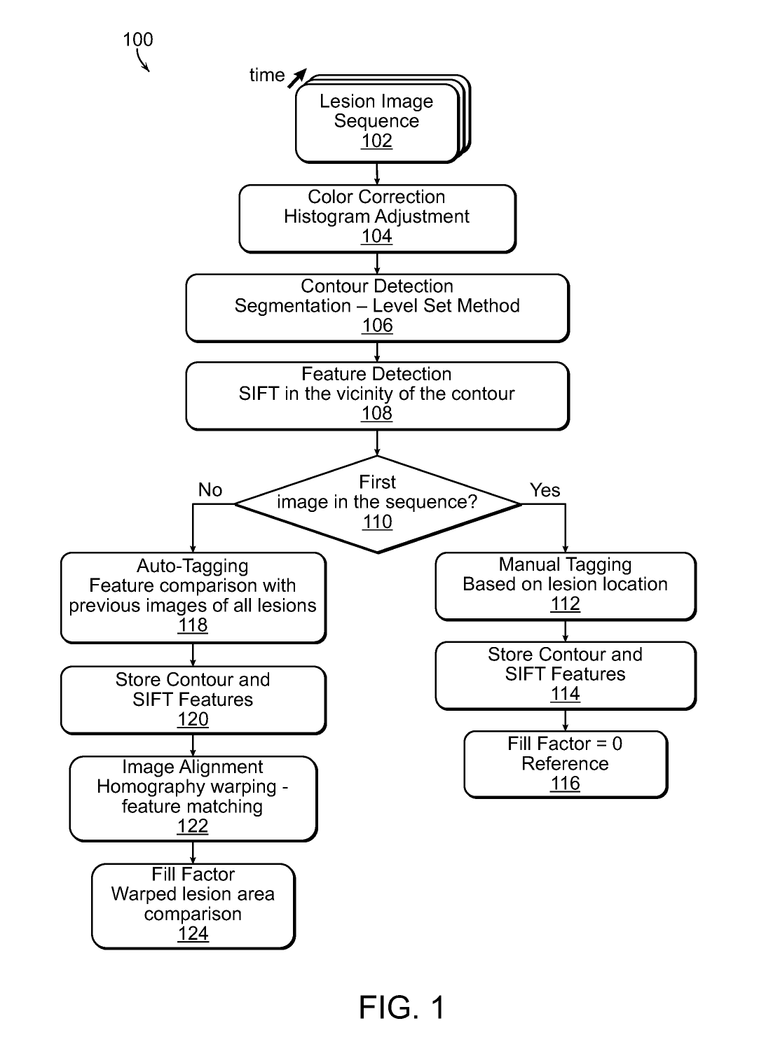

FIG. 1 is a flowchart of an example overall process flow 100 for analyzing skin images for lesions. At step 102, the progress of a skin lesion is recorded by capturing images of the lesion at regular intervals of time. This can be done for all lesions located on different body areas. Color correction is performed at step 104 by adjusting R, G, B histograms to neutralize the effects of varying lighting and to enhance the contrast. A level set method (LSM) based image segmentation approach is used at step 106 to identify the lesion contours. In the vicinity of the lesion contours, Scale Invariant Feature Transform (SIFT) based feature detection is performed at step 108 to identify key features of the lesion. Once a new image is captured, it is registered with the first image in the sequence for that lesion using SIFT feature matching. The warped lesion contours are computed after alignment and their area is compared to the area of the first lesion in the sequence to determine the fill factor that indicates the change in area over time and quantifies the progress over time. Steps 104, 106, and 108 of method 100 are described in detail below. Preferred embodiments thus are operative to computationally compensate for the change in alignment that can occur during image acquisition as different analyses relative to a body region such as a wound, or mole or a skin lesion.

Accurate color information of skin lesions is significant for dermatology diagnosis and treatment. However, different lighting conditions and non-uniform illumination during image capture often lead to images with varying color profiles. Having a consistent color profile in the images captured over time is important for both visual comparison as well as to accurately determine the progression over time. Thus, methods for color correction for 104 of acquired images can be performed in accordance with preferred embodiments. Some embodiments normalize the color profile of the instruments to match the images captured with different devices through users characterizing and calibrating the color response for dermascopic instruments. Alternative embodiments for the color correction module 104 use color normalization filters by analyzing features in a large data set of images for a skin condition, which extracts image features from the inside, outside, and peripheral regions of the tumor and builds multiple regression models with statistical feature selection. The preferred embodiment uses a color correction scheme that automatically corrects for color variations and enhances image contrast using color histograms. Performing histogram equalization on R, G and B color channels independently, brings the color peaks in alignment and results in an image that closely resembles one in neutral lighting environment. For an image I, the color histogram for channel c (R, G or B) is modified by adjusting the pixel color values I_{c}(x, y) to span the entire dynamic range D, as given by equation 1 below.

$${I}_{c}^{M} ( x , y ) = \frac{{I}_{c} ( x , y ) - {I}_{c}^{l}}{( {I}_{c}^{u} - {I}_{c}^{l} )} \times D \qquad ( 1 )$$

where, I_{c}^{u} and I_{c}^{l} represent the upper and lower limits of the histogram. The color correction process can be summarized in the following steps. First, the histograms for R, G and B color channels are computed. In the second step, the upper and lower limits of the R, G and B histograms are determined as the +2τ limit (I_{c}^{u}≧intensity of 97.8% pixels) and the −2τ limit (I_{c}^{l}≦intensity of 97.8% pixels). Such limits can avoid histogram skewing due to long tails and results in better peak alignment. In the final step, the R, G, B histograms are expanded to occupy the entire dynamic range (D) of 0 to 255 by modifying pixel color values using equation 1.

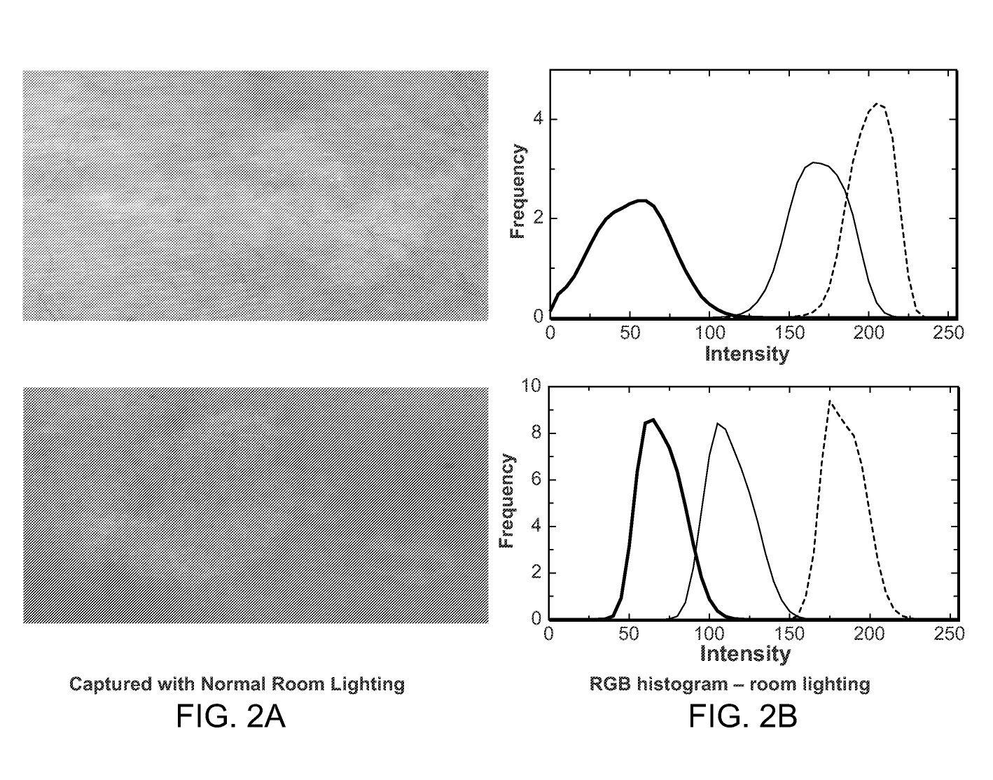

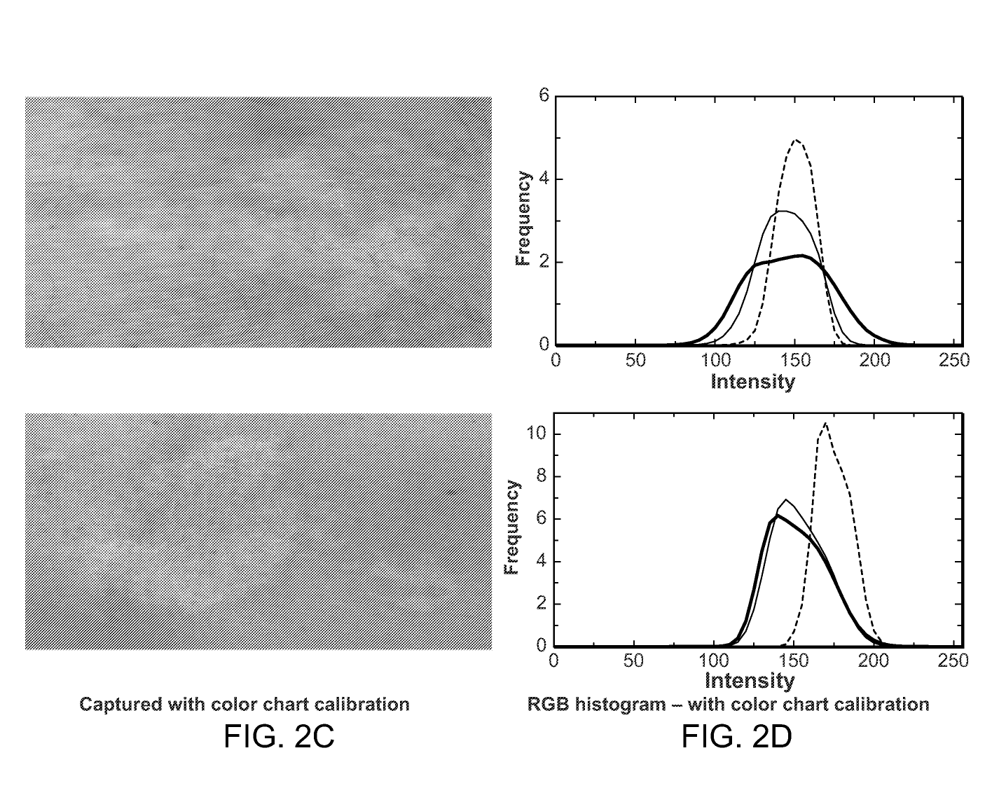

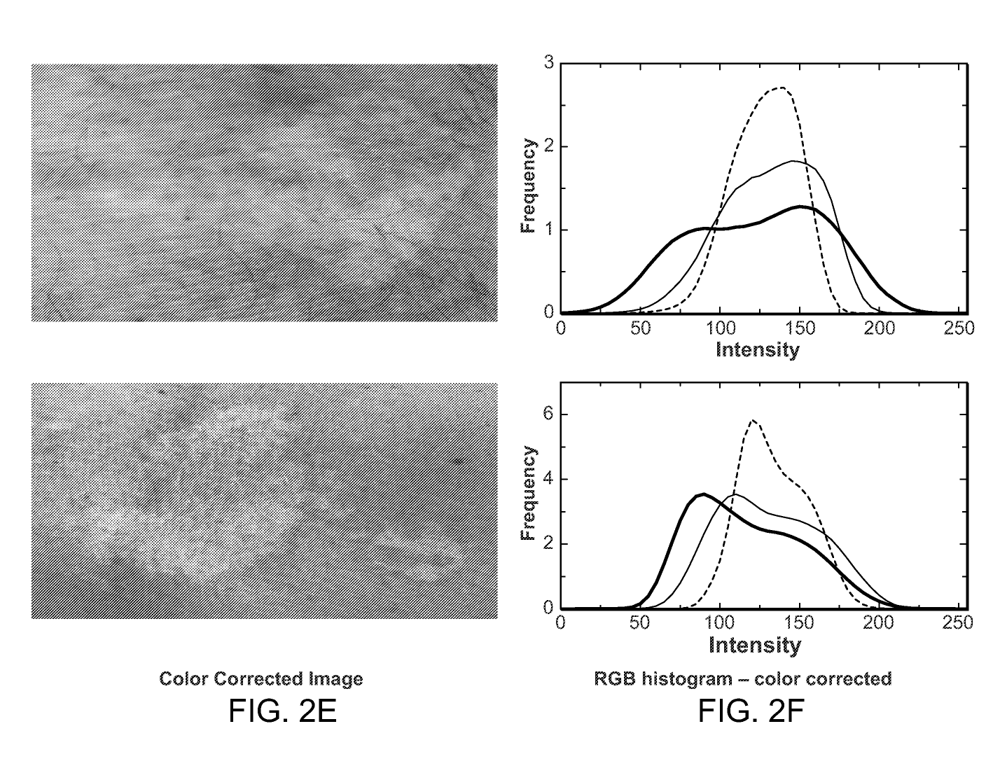

FIG. 2 shows images of two different skin lesions where the color correction by histogram matching process has been performed on the images. The color corrected images exhibit similar qualities to those captured by white-balance calibration with a color chart and enhance contrast to make the lesions more prominent. FIG. 2(a) are images captured with normal room lighting. FIG. 2(b) are R, G, B histograms of images captured with room lighting shown in FIG. 2(a). FIG. 2(c) are images captured with color chart white-balance calibration. FIG. 2(d) are R, G, B histograms of images with color chart calibration shown in FIG. 2(c). FIG. 2(e) are images after the color correction and contrast enhancement described above are performed on the images shown in FIG. 2(a). FIG. 2(f) are R, G, B histograms of the images shown in FIG. 2(e). The image can also be a plurality of images of a single body that are segmented for processing and stitched together for diagnostic analysis. A first image of the entire feature can be taken followed by separate images of each sector of the feature which account for the size and/or shape of the feature.

Accurately determining the contours of skin lesions with a contour detection module 106 can aid in diagnosis and treatment as the contour shape is often a feature used in determining the skin condition. Contour shape is also important for determining the response to treatment and the progress over time. Due to non-uniform illumination, skin curvature, and camera perspective, the images tend to have intensity and color variations within lesions. This makes it difficult for segmentation algorithms that rely on intensity or color uniformity to accurately identify the lesion contours. Some embodiments use a level set approach that models the distribution of intensity belonging to each tissue as a Gaussian distribution with spatially varying mean and variance, and creates a level set formulation by defining a maximum likelihood objective function. Alternative embodiments use a level set method (LSM) based approach called the distance regularized level set evolution (DRLSE) or a region-based image segmentation scheme that can take into account intensity inhomogeneities. Based on a model of images with intensity inhomogeneities, the region-based image segmentation scheme derives a local intensity clustering property of the image intensities, and defines a local clustering criterion function for the image intensities in a neighborhood of each point. In a preferred embodiment, the level set method using the region-based image segmentation scheme is used, and developed for a narrowband implementation. The image with non-uniform intensity profile is modeled by equation 2.

I=bJ+n (2)

where, J is the image with homogeneous intensity, b represents the intensity inhomogeneity, and n is the additive zero-mean Gaussian noise. The segmentation partitions the image into two regions Ω₁ and Ω₂ that represent the skin lesion and the background respectively. The true image J is represented by two constants c₁ and c₂ in these regions. A level set function (LSF) φrepresents two disjoint regions Ω₁ and Ω₂ as given by equation 3.

Ω₁={x:φ(x)>0}, Ω₂={x:φ(x)<0} (3)

The optimal regions Ω₁ and Ω₂ are obtained by minimizing the energy, F (φ, {c₁,c₂},b), in a variational framework defined over Ω₁, Ω₂, c₁, c₂, and b. The energy minimization is performed in an iterative manner with respect to one variable at a time while the other variables are set to their values in the previous iteration. The iterative process is implemented numerically using a finite difference method.

The narrowband implementation is achieved by limiting the computations to a narrow band around the zero level set. The LSF at a pixel (i, j) in the image is denoted by φ_{i,j} and a set of zero-crossing pixels is determined as the pixels (i, j) such that either φ_{i+1,j} and φ_{i-1,j}, or φ_{i,j+1} and φ_{i,j-1} have opposite signs. If the set of zero crossing pixels is denoted by Z, the narrowband B is constructed as given by equation 4 below.

$$B {⋃}_{( i , j ) ∈ Z} {N}_{i , j} \qquad ( 4 )$$

where, N_{i,j} is a 5×5 pixel window centered around pixel (i, j). In a preferred embodiment, the 5×5 window is measured to provide a good trade-off between computational complexity and quality of the results. The LSF based segmentation using narrowband can be summarized by the following steps. First, the LSF is initialized to φ_{i,j}⁰, where φ_{i,j}^{k} indicates the LSF value during iteration k. The narrowband B⁰ is constructed using equation 4. Next, the LSF is updated on the narrowband using a finite difference scheme as φ_{i,j}^{k+1}=φ_{i,j}^{k}+Δt·L(φ_{i,j}^{k}), where Δt is the time step of the iteration and

$$L ( {φ}_{i , j}^{k} ) \approx \frac{\partial φ}{\partial t} .$$

In the third step, the set of zero-crossing pixels of φ_{i,j}^{k+1} is determined, and the narrowband B^{k+1} is updated using equation 4. Next, for pixels (i, j) part of the updated narrowband B^{k+1} that were not part of the narrowband B^{k}, values are set according to φ_{i,j}^{k+1}=3 if φ_{i,j}^{k+1}≧0, and φ_{i,j}^{k+1}=−3 otherwise. In the final step, iterations are continued until the narrowband stops changing (B^{k+1}=B^{k}=B^{k-1}) or the limit on maximum iterations is reached. The set of zero-crossing points at the end of iteration represents the segmentation contour.

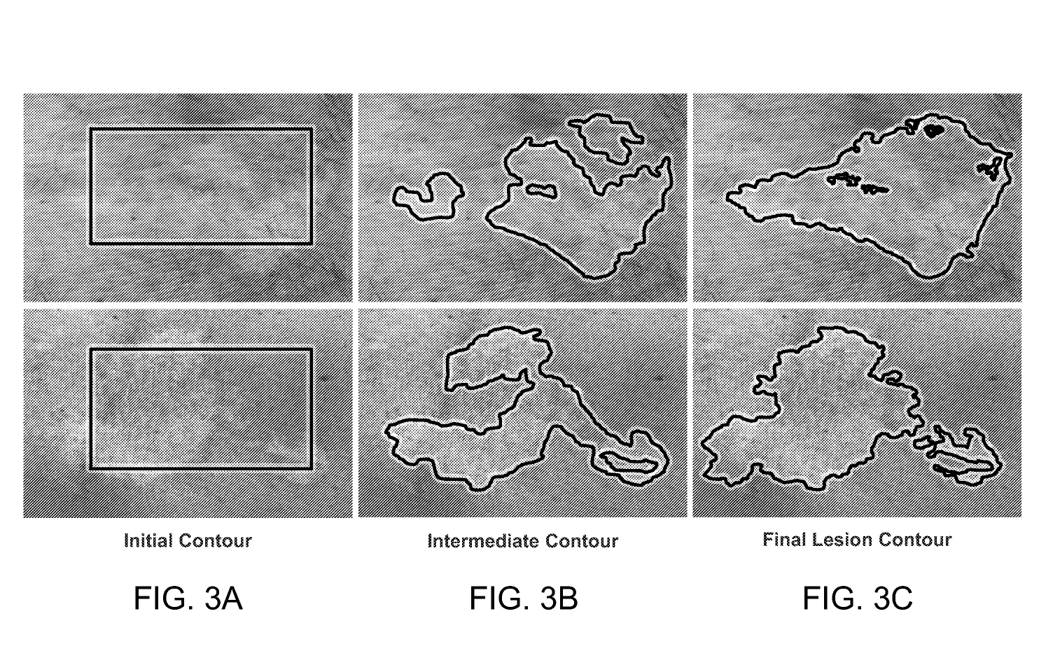

The segmentation approach is applied for contour detection to clinical images of skin lesions. FIG. 3 shows the evolution of the contours for two different skin lesions. The shared region around the contour defines the narrowband used for LSF update.

The images in FIG. 3 illustrate the determining of lesion contours using the example segmentation mechanism described above. FIG. 3(a) shows the initial contours of two images. FIG. 3(b) shows the intermediate contours of two images. FIG. 3(c) shows the final segmented contours for images of the two lesions. The shaded region around the contour defines the narrowband used for LSF update described above.

The ability to accurately determine the progression of a skin condition over time is an important aspect of diagnosis and treatment. In some embodiments, images of the same skin lesions can be captured using a handheld digital camera over an extended period of time during treatment. These images can be analyzed to determine the progress of the disease or treatment. The lesion contours determined in individual images cannot be directly compared as the images typically have scaling, orientation and perspective mismatch. Thus, a feature detection module 108 can be used to measure quantitative geometric characteristics of lesions as a function of time.

In a preferred embodiment, an image registration method based on Scale Invariant Feature Transform (SIFT) feature matching is used for progression analysis. Skin surfaces typically do not have significant features that can be detected and matched across images by SIFT. However, the lesion boundary creates distinct features due to transition in color and intensity from the regular skin to the lesion. To further highlight these features, the identified contour is superimposed on to the original image before feature detection. The lesion contours change over time as the treatment progresses, however this change is typically slow and non-uniform. Repigmentation often occurs within the lesion and some parts of the contour shrink while others remain the same. Performing SIFT results in several matching features corresponding to the areas of the lesion that have not significantly changed. In some embodiments, matching SIFT features over large images can be computationally expensive. In such cases, the feature matching using SIFT is restricted to a narrow band of pixels in the neighborhood of the contour, defined in the same manner as the narrow band in equation 4. This significantly speeds up the processing, while providing significant features near the contour that can be matched across images. In a preferred embodiment, SIFT is performed only once on any given image, the first time 110 it is analyzed. Note that manual tagging 112 or auto-tagging 118 can be used. At modules 114 and 120, the SIFT features for the image are stored in a database and can be used for subsequent analyses.

Once the SIFT features are determined in all the images in a sequence, matching features are identified across images using random sample consensus (RANSAC). Based on the locations of the matching features, homography transforms are computed at module 122 that map every image in the sequence to the first image and the images are warped or transformed to align with the first image in the sequence.

Many skin conditions typically result in lesions in multiple body areas. For a patient or a doctor to be able to keep track of the various lesions, it is important to be able to classify the lesions based on the body areas. In some embodiments, individual databases are maintained for a sequence of images from each lesion. In a preferred embodiment, the user manually identifies the lesions once, during initial setup at module 112. Then all future instances of the lesion at the same location are automatically classified at module 118, and entered into the database for analysis.

At the beginning of the treatment, all skin lesions are photographed and manually tagged based on the body areas at module 112. An image of lesion i captured at time t is denoted by Lᵢ¹. The images (Lᵢ⁰) are processed to perform color correction and contour detection, as described above with respect to modules 104 and 106. SIFT features are computed for each image as described with respect to block 108, and stored at module 114 along with the image as Sᵢ⁰. When a new image (Lⱼ¹) is captured at time t=1, the same processing (as modules 104, 106, and 108) is performed to determine the contour and SIFT features. The determined contours and features are stored at module 120.

At module 122, the SIFT features for the new image (Sⱼ¹) are compared with those determined earlier (Sᵢ⁰) to find matches using two nearest neighbor approach. The largest set of inliers (I_{i,j}) with N_{i,j} elements and the total symmetric transfer error (e_{i,j}) (normalized over the range [0, 1]) for every combination {Sᵢ⁰, Sⱼ¹} are determined using RANSAC. The image (Lⱼ¹) is then classified to belong to lesion i if the given i maximizes the matching criterion M_{i,j}, defined by equation 6.

M_{i,j}=N_{i,j}(1+λ(1−e_{i,j})) (6)

where, λ is a constant and set to 0.2 in a preferred embodiment. The homography Hᵢ^{0,1}, corresponding to the best match, is stored for later use in progression analysis. The same process is applied for tagging any future image Lⱼⁿ by comparing it against the previously captured set of images Lᵢⁿ⁻¹.

Lesion contours in the warped or transformed images can be used to compare the lesions and determine the progression over time. The lesion area, confined by the warped or altered contours, is determined for each image in the sequence and a quantitative metric called fill factor (F_{T}) at time T is defined as the change in area of the lesion with respect to the reference (first image, for example, captured before the beginning of the treatment, or a later image that is designated as a reference), given by equation 5 below.

$${F}_{T} = 1 - \frac{{A}_{T}}{{A}_{0}} \qquad ( 5 )$$

where A_{T} is the lesion area at time T and A₀ is the lesion area in the reference image. If this is the first image in the sequence, then the fill factor value is stored as 0 at module 116.

In this manner systems and methods are provided for image tagging, lesion contour detection and progression analysis of skin diseases. In summary, the initial setup includes manual tagging by user of images (Lᵢ⁰) based on the location i of the lesion. Then, color correction and image segmentation is performed to determine lesion contours (Cᵢ⁰), and SIFT features (Sᵢ⁰) in the vicinity of the lesion contour (Cᵢ⁰) are computed. The contours and features are stored as Cᵢ⁰ and Sᵢ⁰ for future analysis.

The subsequent analysis includes performing color correction and contour detection (Cⱼ^{t}) for an image Lⱼ^{t} captured at time t, and computing SIFT features (Sᵢ^{t}) in the vicinity of the lesion contour (Cⱼ^{t}). Next, feature matching 122 is performed for every combination {Sᵢⁱ⁻¹, Sⱼ^{t}} and tagged as Lⱼ^{t} to lesion i using equation 6 above. The best match homography Hᵢ^{t-1, t} is stored for further analysis. Using the pre-computed contours (Cᵢ^{t}) and homographies (Hᵢ^{t-1, t}), a sequence of n images of the same lesion captured over time are registered or associated to the first image (Lᵢ⁰). The areas of the warped lesion contours are compared to determine the progression over time and compute the fill factor 124 (Fᵢ^{t}) using equation 5.

In an example embodiment, the systems and methods described herein to analyze individual images and determine progress of skin lesions over time can be implemented using MATLAB or other suitable programming tool.

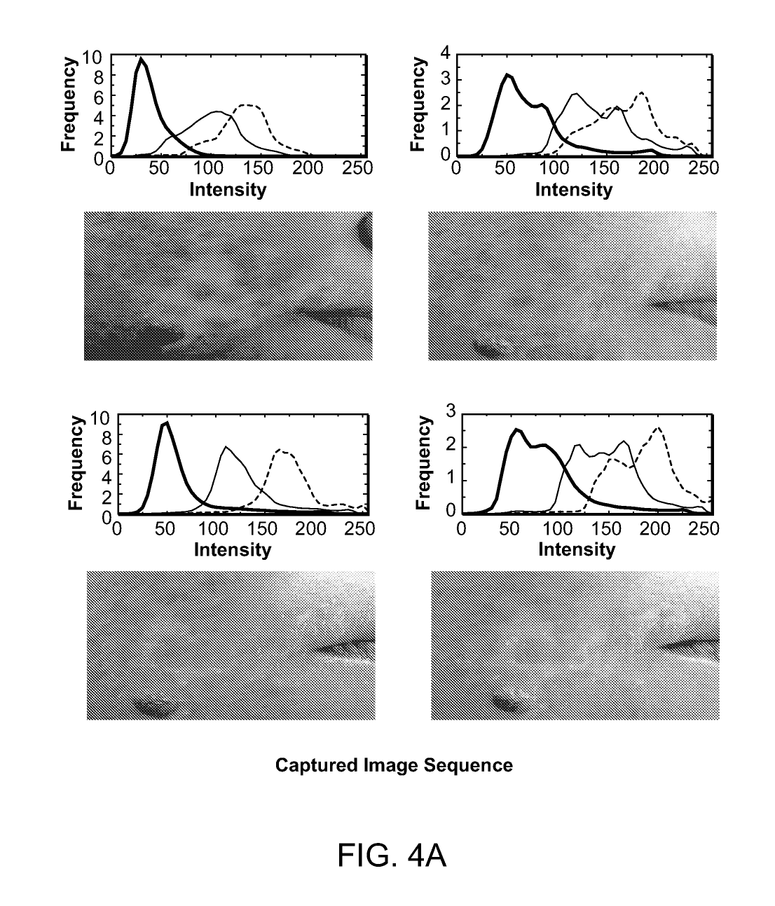

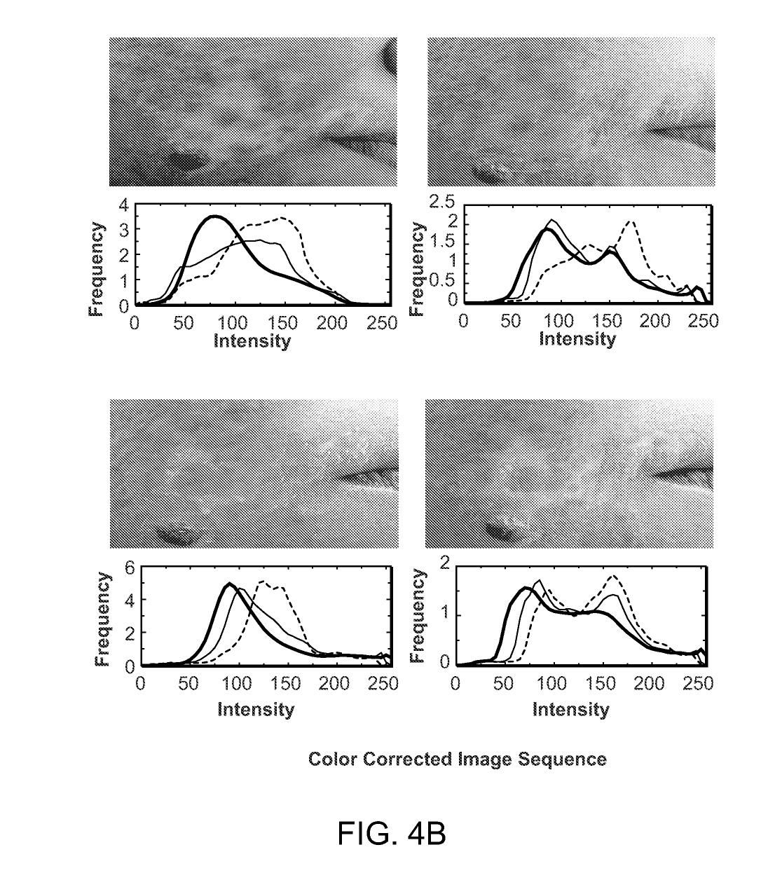

For a sequence of images of a skin lesion captured over time, each image is processed to perform color correction and contrast enhancement. FIG. 4 shows a sequence of images with their R, G, B histograms and the outputs after color correction. FIG. 4(a) shows the original image sequence. FIG. 4(b) shows the color corrected image sequence. The lesion color can change due to phototherapy.

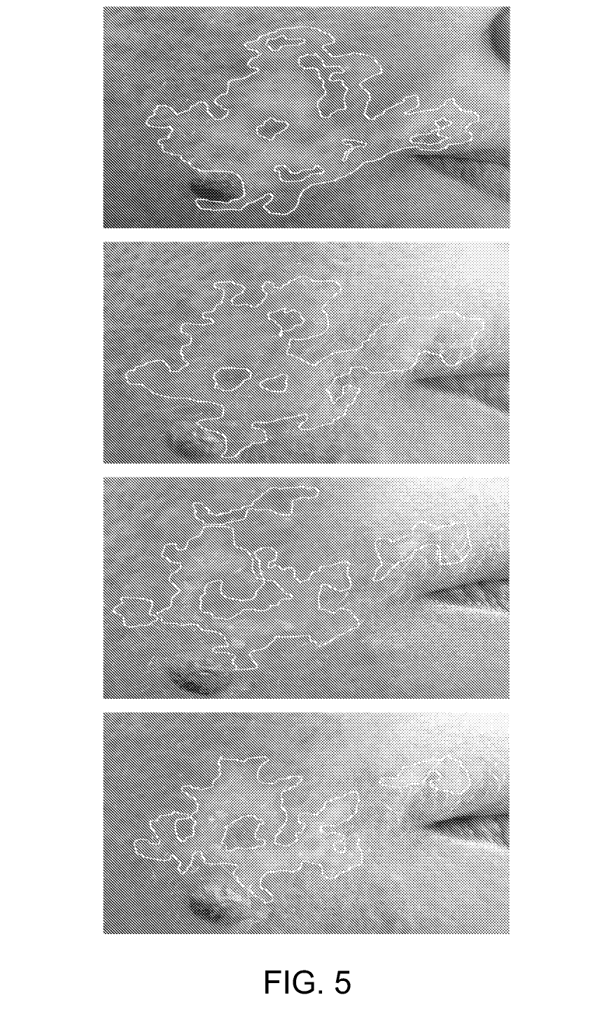

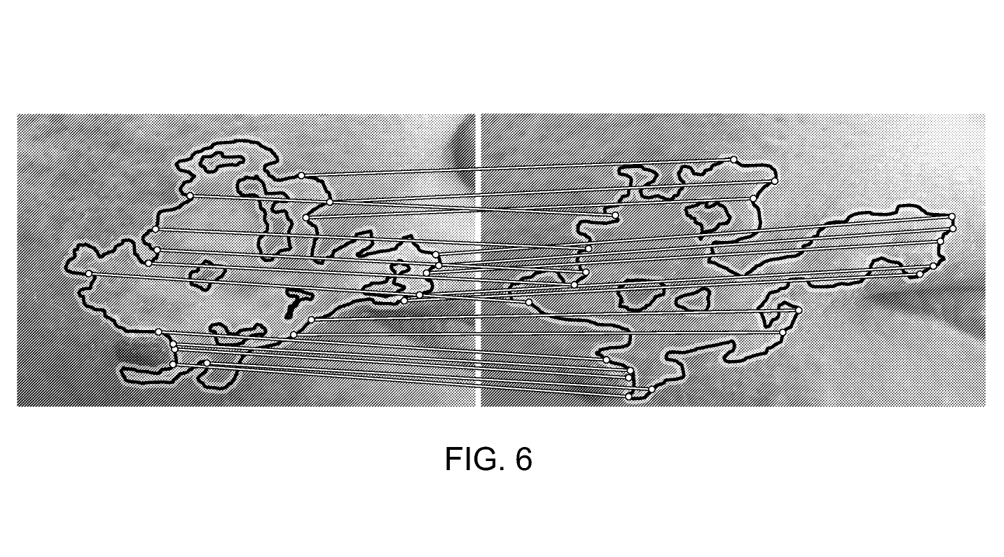

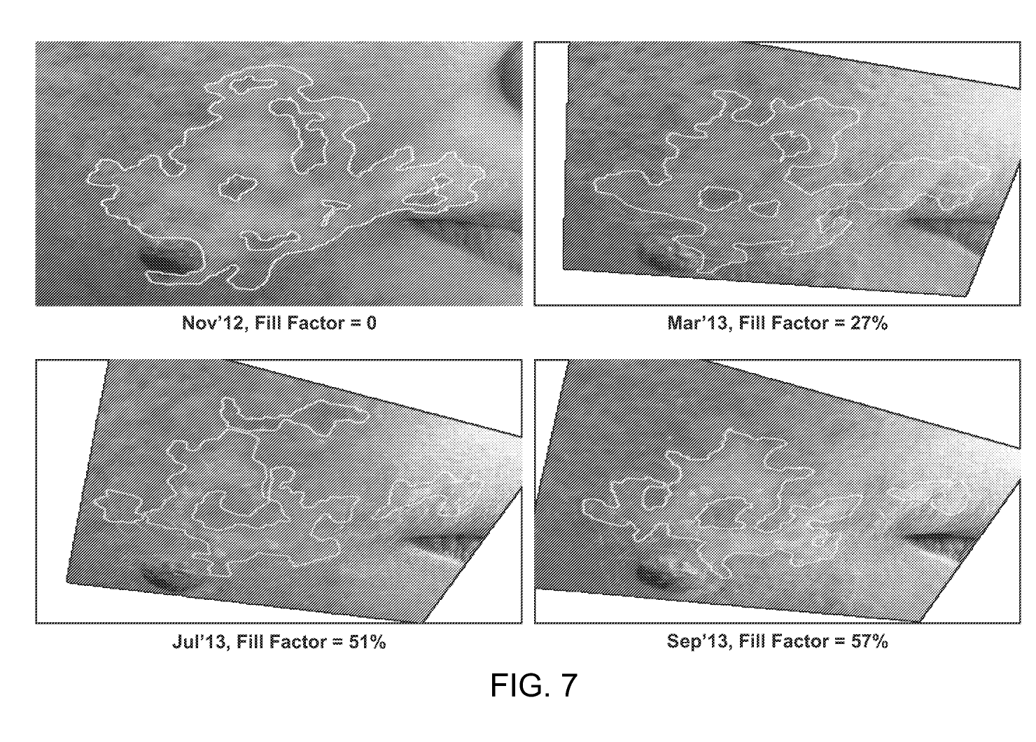

The color corrected images are then processed to perform lesion contour detection. FIG. 5 shows a sequence of image segmentations using LSM for lesion contour detection. LSM based image segmentation accurately detects the lesion boundaries despite intensity or color inhomogeneities in the image. Feature matching is performed across images to correct for scaling, orientation and perspective mismatch. FIG. 6 shows a pair of images of the same lesion with some of the matching SIFT features identified on them. In this example, SIFT feature matching is performed on the narrow band of pixels, highlighted in the figure, in the neighborhood of the lesion contours. An homography transform, the transform being computed based on the matching features, is used to alter all the images in a sequence with respect to the reference image. FIG. 7 shows a sequence of image registrations based on matching features with respect to the reference image at the beginning of treatment. The altered lesion images are compared with respect to the reference lesion image at the beginning of the treatment to determine the progress over time in terms of the fill factor.

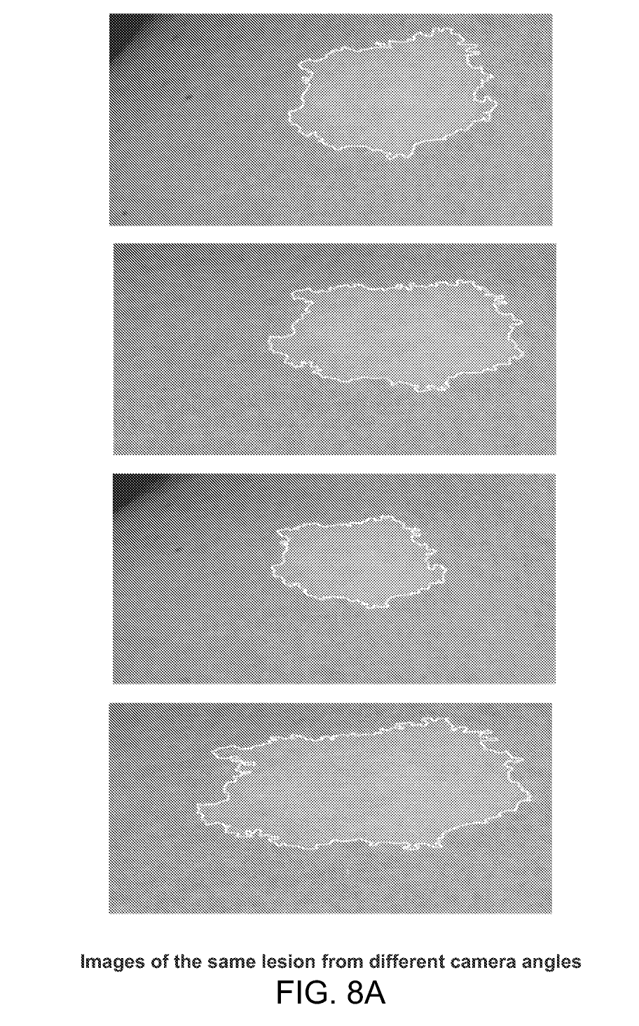

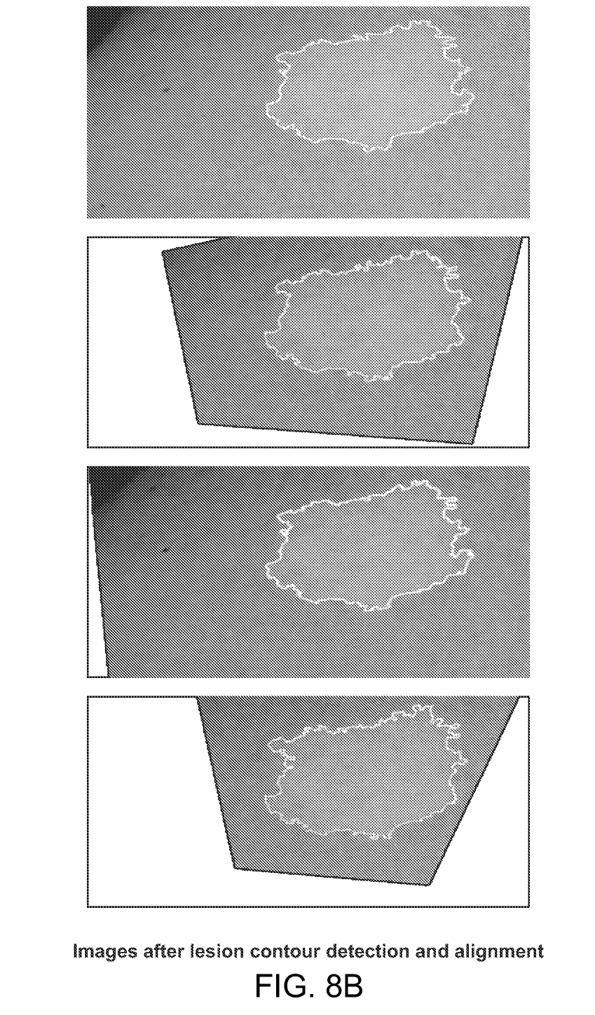

In an example embodiment, image registration is performed by analyzing images of the same skin lesion captured from different camera angles. Contour detection is performed on the individual images that are then aligned by feature matching. FIG. 8 shows one such comparison. FIG. 8(a) shows images of a lesion from different camera angles. FIG. 8(b) shows images after contour detection and alignment. The aligned lesions are compared in terms of their area as well as the number of pixels that overlap. In this example, with four images shown in FIGS. 8(a) and 8(b), area matches to 98% accuracy and pixel overlap to 97% accuracy. In another example, analysis of 100 images from 25 lesions, with four real and artificial camera angles each, shows a 96% accuracy in area and 95% accuracy in pixel overlap.

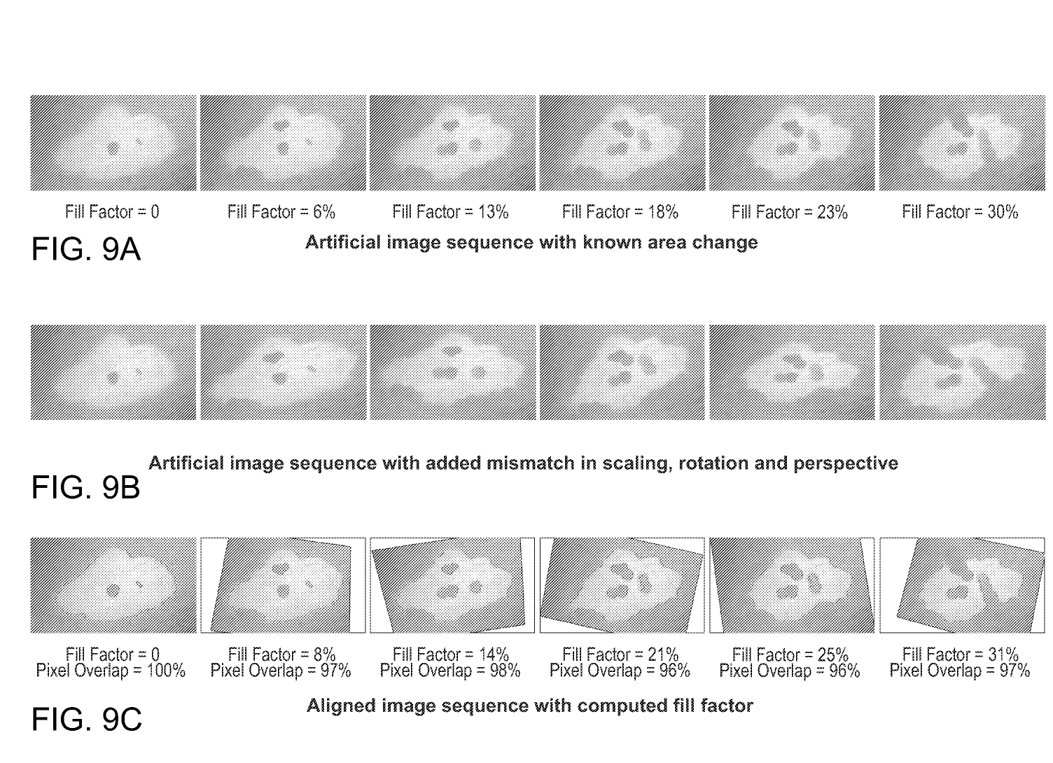

To verify the progression analysis, in one example, a sequence of images is generated for each lesion with known change in area. Rotation, scaling and perspective mismatch is applied to the new images. This sequence is then used as an input to the system described herein to determine the lesion contours, align the sequence and compute the fill factor. The fill factor was with the known change in area from the artificial sequence. The pixel overlap was also computed between the lesions identified on the original sequence (before adding mismatch) and those on the processed sequence. FIGS. 9(a)-9(c) show one such comparison. FIG. 9(a) shows the image sequence with known area change, generated from a lesion image. FIG. 9(b) shows an image sequence after applying scaling, rotation, and perspective mismatch. FIG. 9(c) shows an output image sequence after lesion alignment and fill factor computation. Analysis of 100 images from 25 such sequences shows a 95% accuracy in fill factor computation and pixel overlap.

The results of the above example indicate that the lesion segmentation and progression analysis mechanism described herein is able to effectively handle images captured under varying lighting conditions without the need for specialized imaging equipment. R, G, B histogram matching and expansion neutralizes the effect of lighting variations while also enhancing the contrast to make the skin lesions more prominent. LSM based segmentation accurately identifies the lesion contours despite intensity or color inhomogeneities in the image. The narrowband implementation significantly speeds up processing without sacrificing accuracy. Feature matching using SIFT effectively corrects for scaling, orientation and perspective mismatch in camera angles for a sequence of images captured over time and aligns the lesions that can then be compared to determine progress over time. The fill factor provides an objective quantification of the progression with 95% accuracy, representing a significant improvement over the conventional subjective outcome metrics such as the Physician's Global Assessment and VASI.

In this manner, a system is developed for identifying skin lesions and determining the progression of the skin condition over time. The system is applied to clinical images of skin lesions captured using a handheld digital camera during the course of the phototherapy treatment. The color correction method normalizes the effect of lighting variations. Lesion contours are identified using LSM based segmentation and a registration method is used to align a time sequence of images for the same lesion using SIFT based feature matching. A quantitative metric called fill factor, determined by comparing areas of lesions after alignment, objectively describes the progression of the skin condition over time. Validation on clinical images shows 95% accuracy in determining the fill factor. Thus, this system provides a significant tool for accurate and objective assessment of the progress with impact on patient compliance. The precise quantification of progression enables physicians to perform an objective follow-up study and test the efficacy of therapeutic procedures for best outcomes.

Some embodiments include a portable imaging module that can be used to take images of lesions for analysis as described herein. Medical imaging techniques are important tools in diagnosis and treatment of various skin conditions, including skin cancers such as melanoma. Defining the true border of skin lesions and detecting their features are critical for dermatology. Imaging techniques such as multi-spectral imaging with polarized light provide non-invasive tools for probing the structure of living epithelial cells in situ without need for tissue removal. Light polarization also makes it possible to distinguish between single backscattering from epithelial-cell nuclei and multiple scattered light. Polarized light imaging gives relevant information on the borders of skin lesions that are not visible to the naked eye. Many skin conditions typically originate in the superficial regions of the skin (epidermal basement membrane) where polarized light imaging is most effective.

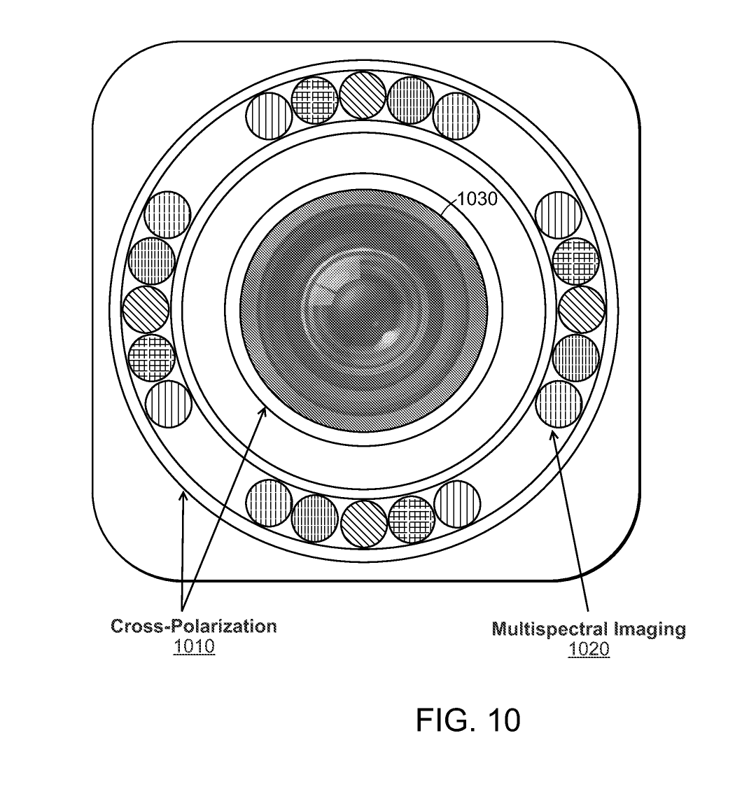

FIG. 10 is a diagram of a preferred portable imaging module with a light source to generate multispectral and/or polarized light for medical imaging. In a preferred embodiment, the portable imaging module is configured to attach or couple to a user's device, such as, a mobile phone or any other hand-held device. The portable imaging module may communicate with the user's device through a wired or wireless connection. The portable imaging module includes an array of lights including a cross-polarization element 1010, and a multispectral imaging element 1020, as shown in FIG. 10, that provide appropriate lighting conditions for capturing images of skin lesions. The array of light elements or sources can comprise Light Emitting Diodes (LEDs) of varying wavelengths, such as infrared, the visible spectrum and ultraviolet, to create lighting conditions for multi-spectral photography. The light sources can be trigged one at a time or simultaneously in response to control signals from the user's device or via a mechanism independent of the user device. The portable imaging module may have a circular shape and may have an aperture in the center of the module, so that it can be attached to the user device around a camera 1030 on a user device. Typical cameras on mobile phones are of circular shape and small size, and the portable imaging module can be configured to couple light returning from a region of interest on the tissue of a patient to the camera aperture on the device. Other devices may have cameras with varying shapes and sizes, in that case, the portable imaging module may have a shape and size that fits around such cameras. Often mobile devices have two cameras—a front-facing and a back-facing. The portable imaging device may be capable of attaching to either camera on the mobile device.

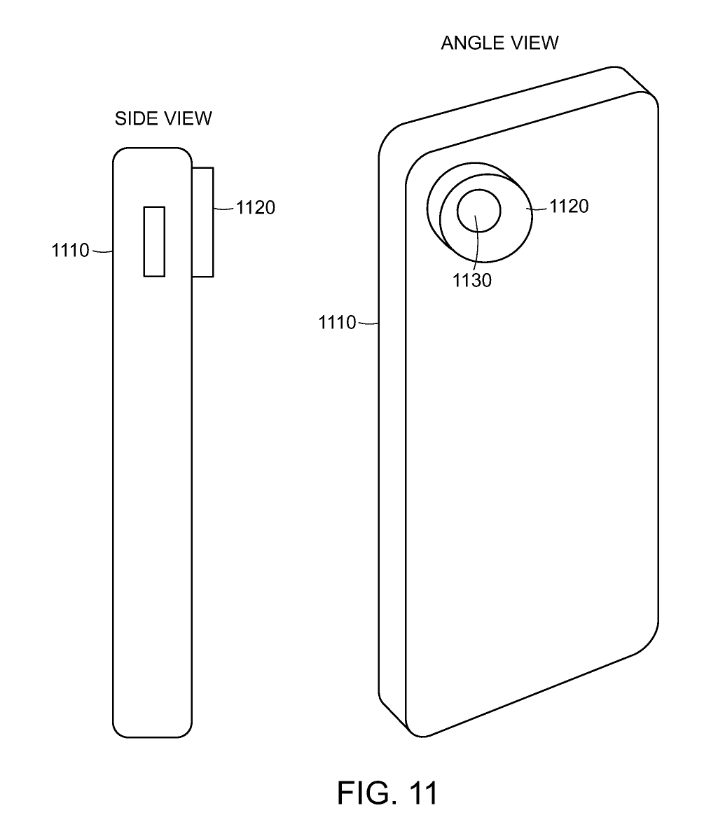

FIG. 11 is a schematic of a side view and an angle view of a portable imaging module mounted on a mobile device. As shown in FIG. 11, portable imaging module 1120 is mounted on mobile device 1110. Mobile device 1110 includes an imaging device, such as camera 1130 having at least 1 million pixels. As shown, portable imaging module 1120 fits around camera 1130 of mobile device 1110. The module 1120 or housing can have a separate controller linked to the mobile device, can utilize a second battery to power the light source, can be motorized to alter the polarization state of light delivered to, or collected from, the body feature being imaged and can include a separate control panel to activate operation or set programmable features of the detachable module 1120. The housing 1120 can include an electrical connector to enable electrical communication between the components. Where the mobile device comprises a web enabled mobile phone, remote commands can be delivered to the composite imaging device.

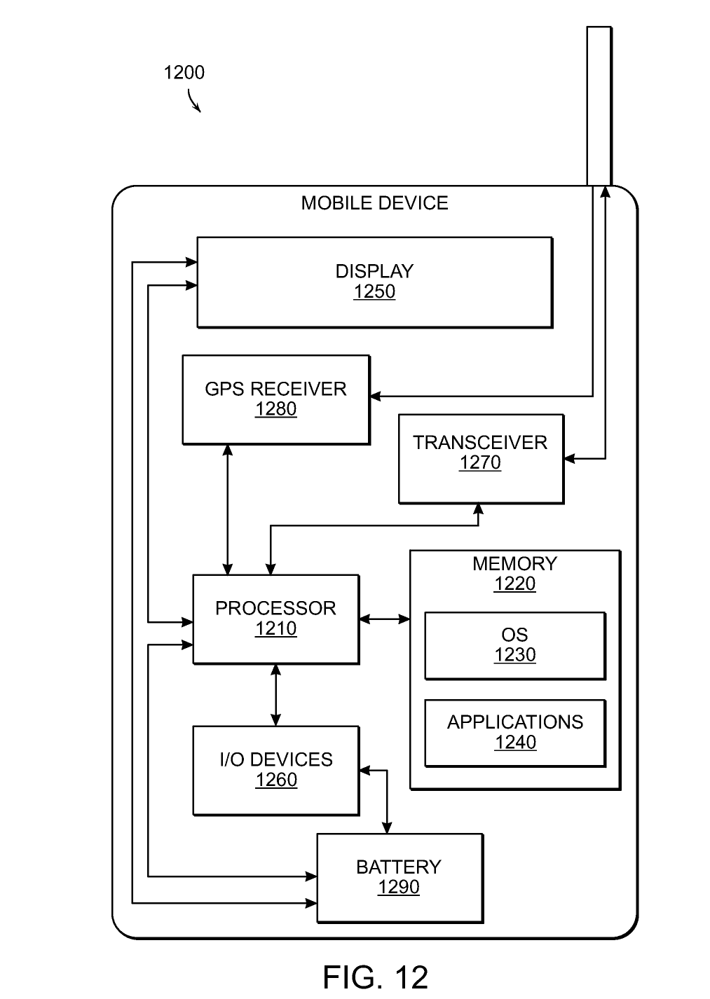

FIG. 12 is a block diagram illustrating a mobile device for implementing systems and methods associated with a workflow, evaluation and grading application, according to an example embodiment. In an example embodiment, the mobile device 1200 includes one or more processor(s) 1210, a memory 1220, I/O devices 1260, a display 1250, a transreceiver 1270, a GPS receiver 1280, and a battery 1290. The processor(s) 1210 may be any of a variety of different types of commercially available processors suitable for mobile devices (for example, XScale architecture microprocessors, Intel® Core™ processors, Intel® Atom™ processors, Intel® Celeron® processors, Intel® Pentium® processors, Qualcomm® Snapdragon processors, ARM® architecture processors, Microprocessor without Interlocked Pipeline Stages (MIPS) architecture processors, Apple® A series System-on-chip (SoCs) processors, or another type of processor). The processor(s) 1210 may also include a graphics processing unit (GPU). The memory 1220, such as a Random Access Memory (RAM), a Flash memory, or other type of memory, is accessible to the processor(s) 1210. The memory 1220 can be adapted to store an operating system (OS) 1230, as well as application programs 1240, such as the retinopathy workflow, evaluation, and grading system described herein. The processor(s) 1210 is/are coupled, either directly or via appropriate intermediary hardware, to a (touchscreen) display 65 and to one or more input/output (I/O) devices 1260, such as a manual or virtual keypad, a touch panel sensor, a microphone, and the like. The mobile device 1200 is also capable of establishing Wi-Fi, Bluetooth and/or Near Field Communication (NFC) connectivity. Similarly, in some embodiments, the processor 310 may be coupled to a transceiver 370 that interfaces with an antenna 390. The transceiver 370 may be configured to both transmit and receive cellular network signals, wireless data signals, or other types of signals via the antenna 390, depending on the nature of the mobile device 115. In this manner, the connection 210 with the communication network 220 may be established. Further, in some configurations, a GPS receiver 380 may also make use of the antenna 390 to receive GPS signals. One or more components of mobile device 1200 is operated by battery 1290, or alternatively, using a battery, power regulation circuit and a processor or controller in the module 1120. In some embodiments, the portable imaging module is powered by battery 1290 in mobile device 1200. The portable imaging module may connect to mobile device 1200 to obtain power via Wi-Fi, Bluetooth, or NFC.

In some embodiments, the portable imaging module includes systems and methods for monitoring the progression of skin disease from the images captured using the portable imaging module. The systems and methods for monitoring and analysis may be included or installed on the user's device, for example, as a software application. The systems and methods included on the user device may also control some of the elements of the portable imaging module. The software application may turn on the plurality of LED arrays in a particular sequence. For example, the first LED array may be activated, then after the first one is deactivated, the next LED array may be activated. The software application can include a specific order in which the elements of the portable imaging module are to be activated so that an optimal light setting is provided for taking an image of a skin lesion.

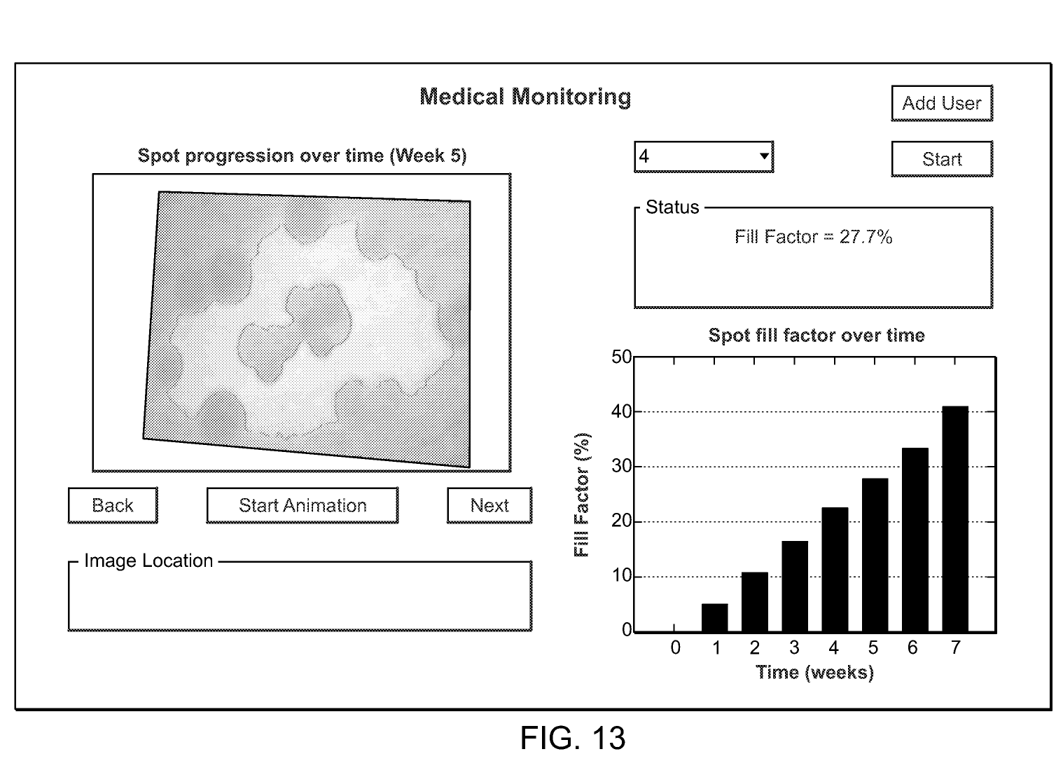

A preferred embodiment includes a software application that can be used with the portable imaging module described herein, or as a standalone application with any other imaging system. The software application includes a graphical user interface (GUI), a patient database, and imaging analysis modules. The GUI may be an intuitive user interface that can be used to add new patients, or analyze the images captured for an existing patient to monitor the progress of their skin condition over time. FIG. 13 shows an example GUI for analysis and monitoring of skin lesions. Adding a new patient using the GUI may create a database for that patient and assign a unique ID to it. All the images taken over time for that patient can be stored in this database. When a new image is captured, it is automatically added to the database and can be transmitted to a remote database such as a data warehouse for stored medical records that can be associated with a clinic or hospital.

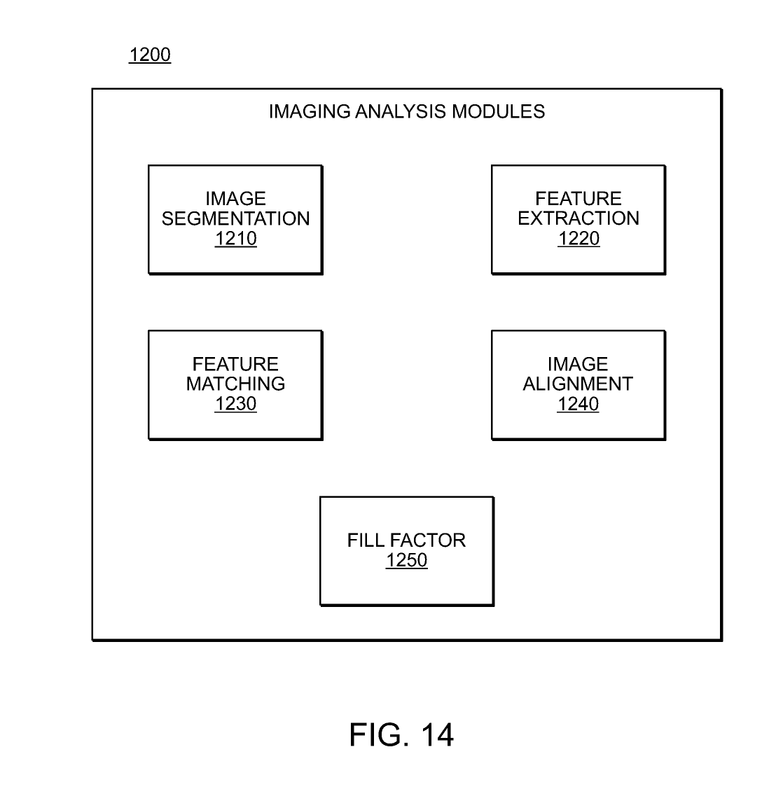

The imaging modules can analyze and monitor the progress of the skin lesions as described herein. FIG. 14 is a block diagram 1200 showing the imaging modules for skin lesion image analysis according to an example embodiment. The modules can be implemented in mobile device 1200 and/or client devices 1410, 1415, 1420, 1425 (as described in further detail herein). The modules can comprise one or more software components, programs, applications, apps or other units of code base or instructions configured to be executed by one or more processors included in client devices 1410, 1415, 1420, 1425. In some embodiments, the modules include an image segmentation module 1210, a feature extraction module 1220, a feature matching module 1230, an image alignment module 1240, and a fill factor module 1250.

The imaging analysis modules may perform any or all of the functionalities described herein. For example, the image segmentation module 1210 can be configured to identify the shape of the depigmented skin lesion in the image. The feature extraction 1220 can be configured to detect the key features in the image using SIFT. The feature matching module 1230 can be configured to perform, for any two consecutive images, Iₙ and Iₙ₊₁, feature matching to identify same areas in the two images. The image alignment module 1240 can be configured to compute a homography to align the two images using matching features, and to warp image Iₙ₊₁ using the homography to align it with image Iₙ. The fill factor module 1250 can be configured to compute the area of the depigmented skin lesion in each aligned image, where the percentage change in area in image Iₙ compared to image I₀ is defined as the fill factor at time n.

In some embodiments, the modules 1210, 1220, 1230, 1240, and 1250 may be downloaded from a web site associated with a health care provider. In some embodiments, the modules 1210, 1220, 1230, 1240, and 1250 may be downloaded as an “app” from an ecommerce site appropriate for the type of computing device. For example, if the client device 1410, 1415, 1420, or 1425 comprises an iOS-type device (e.g., iPhone or iPad), then the modules can be downloaded from iTunes®. Similarly, if the client device 1410, 1415, 1420 or 1425 comprises an Android-type device, then the modules 1210, 1220, 1230, 1240, and 1250 can be downloaded from the Android Market™ or Google Play Store. If the client device 1410, 1415, 1420, or 1425 comprises a Windows® Mobile-type device, then the modules 1210, 1220, 1230, 1240, and 1250 can be downloaded from Microsoft® Marketplace. The modules 1210, 1220, 1230, 1240, and 1250 may be packaged as a skin lesion analysis app. In embodiments for use in areas where internet or wireless service may be unreliable or nonexistent, it may be preferable for all modules to be implemented locally on the client device. Additionally, the modules may include an application programming interface (API) specifying how the various modules of the skin lesion analysis app interact with each other and with external software applications.

In other embodiments, one or more of modules 1210, 1220, 1230, 1240, and 1250 may be included in server 1435 or database server(s) 1440 while other of the modules 1210, 1220, 1230, 1240, and 1250 are provided in the client devices 1410, 1415, 1420, 1425. Although modules 1210, 1220, 1230, 1240, and 1250 are shown as distinct modules in FIG. 12, it should be understood that modules 1210, 1220, 1230, 1240, and 1250 may be implemented as fewer or more modules than illustrated. It should be understood that any of modules 1210, 1220, 1230, 1240, and 1250 may communicate with one or more external components such as databases, servers, database server, or other client devices.

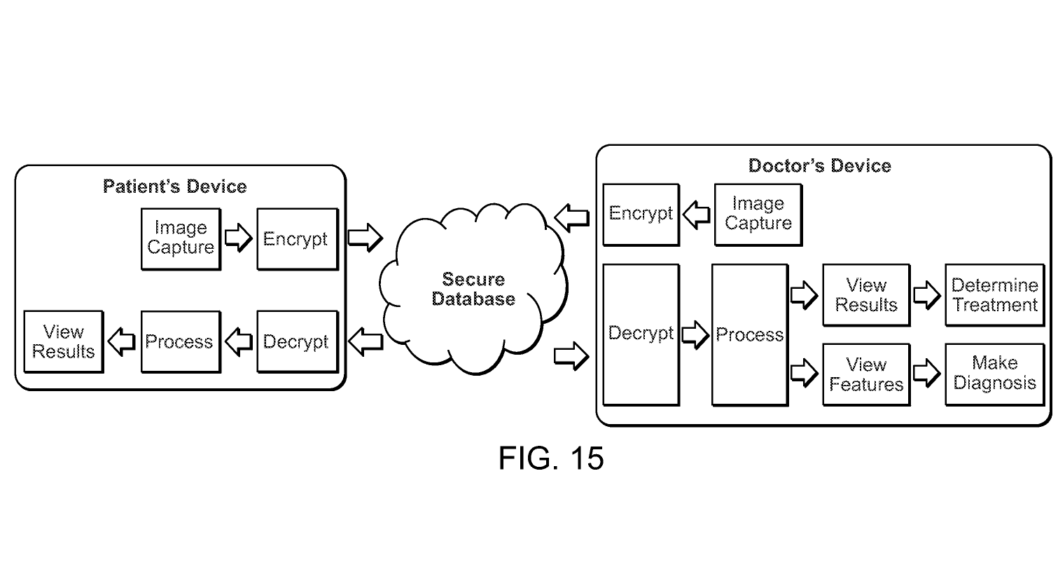

In an example embodiment, a cloud-based secure database can be used to transfer images and information between devices, while the devices locally process the images and information. FIG. 15 is a schematic of a cloud-based secure storage system for securely analyzing and transferring images and patient data. In this embodiment, data processing and analysis occurs on the patient's or doctor's device. The image is encrypted on the patient's device and securely stored in a cloud-database. The image is decrypted on the doctor's device for processing and analysis on the device. After diagnosis and treatment are determined on the doctor's device, the results are encrypted and securely stored in the cloud-database. The patient's device receives the results and decrypts them for the patient's viewing. The shared cloud-database is securely accessible by the patient's device and the doctor's device.

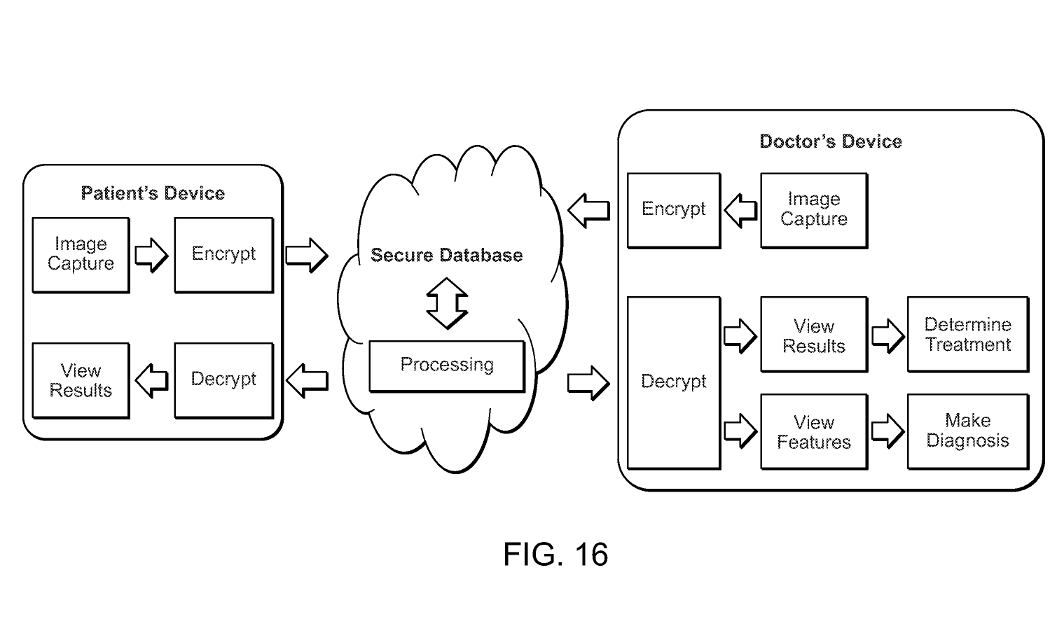

In an alternative embodiment, a cloud-based processing platform processes the images and information, while the devices merely capture, encrypt, and decrypt images and information. FIG. 16 is a schematic of a cloud-based processing platform for securely analyzing and transferring images and patient data. In this embodiment, data processing and analysis occurs within the cloud-based processing platform, rather than the devices. The image is encrypted on the patient's device and sent to the processing platform for processing and analysis. The results, such as contour, features and fill factor determinations, are encrypted and sent to the doctor's device. The doctor's device decrypts the results so that the doctor can make a diagnosis and treatment determination. The cloud-based processing platform provides secure storage and real-time processing.

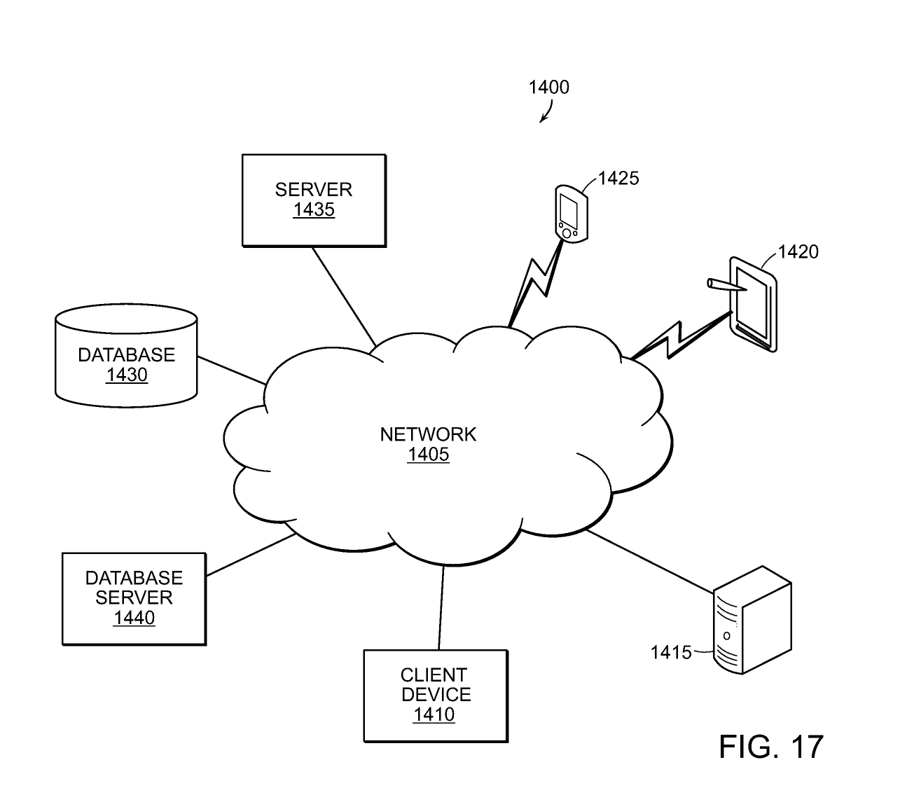

FIG. 17 illustrates a network diagram depicting a system 1400 for a skin lesion analysis system according to an example embodiment. The system 1400 can include a network 1405, a client device 1410, a client device 1415, a client device 1420, a client device 1425, a database(s) 1430, a server 1435, and a database server(s) 1440. Each of the client devices 1410, 1415, 1420, 1425, database(s) 1430, server 1435, and database server(s) 1440 is in communication with the network 1405. One or more of the client devices 1410, 1415, 1420, and 1425 may be a device used by a patient (i.e. patient's device), and one or more of the client devices 1410, 1415, 1420, and 1425 may be a device used by a doctor (i.e. doctor's device).

In an example embodiment, one or more portions of network 1405 may be an ad hoc network, an intranet, an extranet, a virtual private network (VPN), a local area network (LAN), a wireless LAN (WLAN), a wide area network (WAN), a wireless wide area network (WWAN), a metropolitan area network (MAN), a portion of the Internet, a portion of the Public Switched Telephone Network (PSTN), a cellular telephone network, a wireless network, a WiFi network, a WiMax network, any other type of network, or a combination of two or more such networks.

In an example embodiment, the client device 1410, 1415, 1420, or 1425 is a mobile client device. Examples of a mobile client device includes, but are not limited to, hand-held devices, wireless devices, portable devices, wearable computers, cellular or mobile phones, portable digital assistants (PDAs), smartphones, tablets, ultrabooks, netbooks, multi-processor systems, microprocessor-based or programmable consumer electronics, mini-computers, smart watches, and the like. In alternative embodiments, the client device 1410, 1415, 1420, 1425 may comprise work stations, personal computers, general purpose computers, Internet appliances, laptops, desktops, multi-processor systems, set-top boxes, network PCs, vehicle installed computer systems, and the like. Each of client devices 1410, 1415, 1420, 1425 may connect to network 1405 via a wired or wireless connection. Each of client devices 1410, 1415, 1420, 1425 may include one or more applications (also referred to as “apps”) such as, but not limited to, a web browser, messaging application, electronic mail (email) application, notification application, photo or imaging application, a skin-lesion analysis application described herein, and the like. In some embodiments, the skin-lesion application included in any of the client devices 1410, 1415, 1420, 1425 may be configured to locally provide a user interface, locally perform the functionalities described herein, and communicate with network 1405, on an as-needed basis, for acquiring data not locally available or for transferring data to a device or component connected to the network 1405 (transfer or send data to other user's devices so that they may view the skin images and/or results of the diagnosis and treatment). The client device 1410, 1415, 1420, 1425 may include various communication connection capabilities such as, but not limited to WiFi, Bluetooth, or Near-Field-Communication NFC devices.

In an example embodiment, the client device 1410, 1415, 1420, 1425 may capture images, process and analyze the images, and display the results of the analysis. Then when a network connection is available, the client devices 1410, 1415, 1420, 1425 may upload the images and the results of the images analyze, and store the data as corresponding to a patient, thus making it available for download and diagnosis by another user such as a doctor.

In some embodiments, each of the database(s) 1430, server 1435, and database server(s) 1440 is connected to the network 1405 via a wired connection. Alternatively, one or more of the database(s) 1430, server 1435, or database server(s) 1440 may be connected to the network 1405 via a wireless connection. Database server(s) 1440 can be (directly) connected to database(s) 1430, or server 1435 can be (directly) connected to the database server(s) 1440 and/or database(s) 1430. Server 1435 comprises one or more computers or processors configured to communicate with client devices 1410, 1415, 1420, 1425 via network 1405. Server 1435 hosts one or more applications or websites accessed by client devices 1410, 1415, 1420, and 1425 and/or facilitates access to the content of database(s) 1430. Database server(s) 1440 comprises one or more computers or processors configured to facilitate access to the content of database(s) 1430. Database(s) 1430 comprise one or more storage devices for storing data and/or instructions for use by server 1435, database server(s) 1440, and/or client devices 1410, 1415, 1420, 1425. Database(s) 1430, server 1435, and/or database server(s) 1440 may be located at one or more geographically distributed locations from each other or from client devices 1410, 1415, 1420, 1425. Alternatively, database(s) 1430 may be included within server 135 or database server(s) 1440.

In an alternative embodiment, the skin lesion application may be a web-based application that can be accessed on client devices 1410, 1415, 1420, 1425 via a web-browser application.

Certain embodiments are described herein as including logic or a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA), an application-specific integrated circuit (ASIC)), or a Graphics Processing Unit (GPU) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired) or temporarily configured (e.g., programmed) to operate in a certain manner and/or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., APIs).

Example embodiments may be implemented in digital electronic circuitry, or in computer hardware, firmware, software, or in combinations of them. Example embodiments may be implemented using a computer program product, for example, a computer program tangibly embodied in an information carrier, for example, in a machine-readable medium for execution by, or to control the operation of, data processing apparatus, for example, a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special purpose logic circuitry (e.g., a FPGA or an ASIC).

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., machine) and software architectures that may be deployed, in various example embodiments.

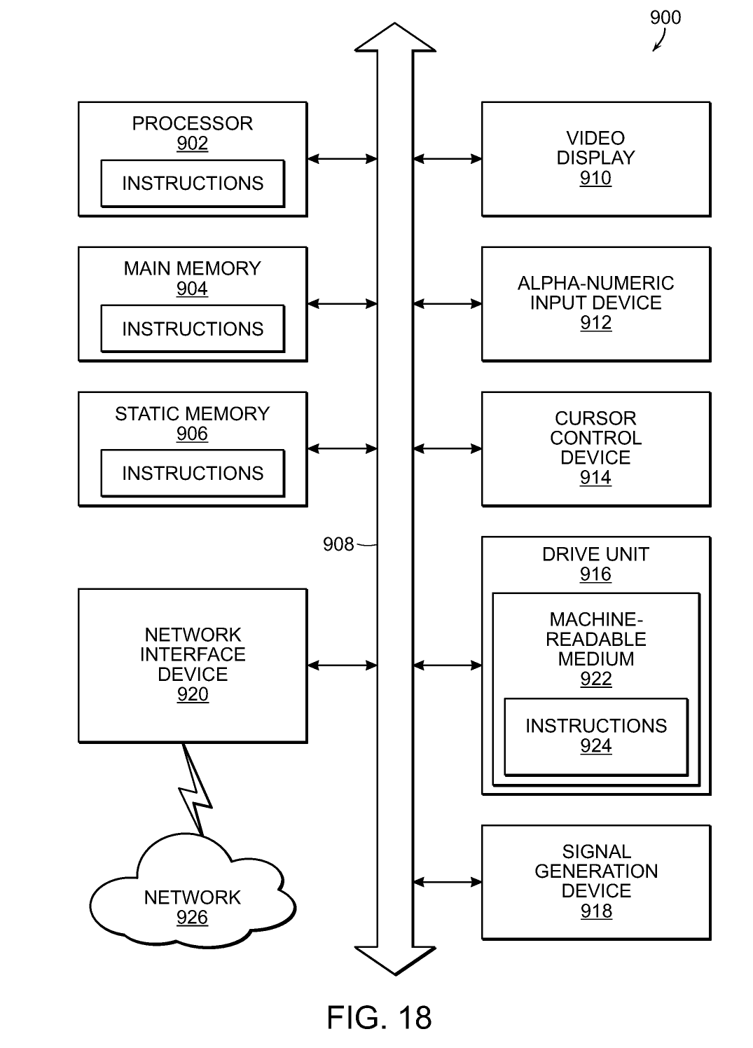

FIG. 18 is a block diagram of machine in the example form of a computer system 900 (e.g., a mobile device) within which instructions, for causing the machine (e.g., client device 1410, 1415, 1420, 1425; server 1435; database server(s) 1440; database(s) 1430) to perform any one or more of the methodologies discussed herein, may be executed. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet, a set-top box (STB), a PDA, a mobile phone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 900 includes a processor 902 (e.g., a central processing unit (CPU), a multi-core processor, and/or a graphics processing unit (GPU)), a main memory 904 and a static memory 906, which communicate with each other via a bus 908. The computer system 900 may further include a video display unit 910 (e.g., a liquid crystal display (LCD), a touch screen, or a cathode ray tube (CRT)). The computer system 900 also includes an alphanumeric input device 912 (e.g., a physical or virtual keyboard), a user interface (UI) navigation device 914 (e.g., a mouse), a disk drive unit 916, a signal generation device 918 (e.g., a speaker) and a network interface device 920.

The disk drive unit 916 includes a machine-readable medium 922 on which is stored one or more sets of instructions and data structures (e.g., software) 924 embodying or used by any one or more of the methodologies or functions described herein. The instructions 924 may also reside, completely or at least partially, within the main memory 904, static memory 906, and/or within the processor 902 during execution thereof by the computer system 900, the main memory 904 and the processor 902 also constituting machine-readable media.

While the machine-readable medium 922 is shown in an example embodiment to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding or carrying instructions for execution by the machine and that cause the machine to perform any one or more of the methodologies of the present invention, or that is capable of storing, encoding or carrying data structures used by or associated with such instructions. The term “machine-readable medium” shall accordingly be taken to include, but not be limited to, solid-state memories, and optical and magnetic media. Specific examples of machine-readable media include non-volatile memory, including by way of example, semiconductor memory devices (e.g., Erasable Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 924 may further be transmitted or received over a communications network 926 using a transmission medium. The instructions 924 may be transmitted using the network interface device 920 and any one of a number of well-known transfer protocols (e.g., HTTP). Examples of communication networks include a LAN, a WAN, the Internet, mobile telephone networks, Plain Old Telephone (POTS) networks, and wireless data networks (e.g., WiFi and WiMax networks). The term “transmission medium” shall be taken to include any intangible medium that is capable of storing, encoding or carrying instructions for execution by the machine, and includes digital or analog communications signals or other intangible media to facilitate communication of such software.

Although the present invention has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense.

It will be appreciated that, for clarity purposes, the above description describes some embodiments with reference to different functional units or processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although an embodiment has been described with reference to specific preferred embodiments, it will be evident that various modifications and changes may be made to these embodiments without departing from the broader spirit and scope of the invention. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be used and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended; that is, a system, device, article, or process that includes elements in addition to those listed after such a term in a claim are still deemed to fall within the scope of that claim. Moreover, in the following claims, the terms “first,” “second,” and “third” and so forth are used merely as labels, and are not intended to impose numerical requirements on their objects.

1. A system for measuring a body condition, the system comprising:
an imaging sensor that detects images of a body region;
a data processor that performs color correction on a first image of the body region, the data processor further performing histogram equalization on a color channel, that performs contour detection on the first image to identify one or more contours of the body region, and that performs feature detection on the first image to identify one or more features of the body region; and
a memory device that stores image data.

2. The system of claim 1, wherein the image sensor is configured to detect a sequence of images wherein the images correspond to a skin lesion represented in the first image, and the sequence of images represent the skin lesion over a period of time.

3. The system of claim 1 wherein the data processor performs color correction on the sequence of images including performing histogram equalization on a color channel; and
the data processor performs contour detection on the sequence of images to identify one or more contours of the skin lesion; and
the data processor performs feature detection on the sequence of images to identify one or more features of the skin lesion.

4. (canceled)

5. (canceled)

6. The system of claim 1 wherein the system stores the resulting sequence of images to record changes from a therapeutic procedure such as phototherapy.

7. The system of claim 2 wherein the data processor determines a progression factor based on a comparison of an area of the skin lesion in the first image and of the skin lesion in the sequence of images.

8. The system of claim 7, wherein the progression factor is determined based on a comparison of the one or more contours of the skin lesion in the first image and the one or more contours of the skin lesion in the sequence of images or wherein the progression factor is determined based on a comparison of the one or more features of the skin lesion in the first image and the one or more features of the skin lesion in the sequence of images.

9. (canceled)

10. The system of claim 1, wherein the color channel comprises a red color channel, a green color channel, and a blue color channel, and the histogram is performed on each of the color channels independently.

11. The system of claim 4, wherein the contour detection comprises performing a level set method using a region-based image segmentation scheme.

12. The system of claim 4, wherein performing the feature detection includes performing a scale invariant feature transform feature matching.

13. The system of claim 1, wherein the system comprises a handheld mobile device to detect a sequence of images.

14. The system of claim 7, wherein a sequence of images are received at a first user device, and the progression factor is determined at the first user device or a second user device.

15. The system of claim 1 wherein the system further comprises a wireless transmitter that sends a sequence of transformed images and the progression factor to a second user device; and
receives, at a first user device with a wireless receiver, diagnosis and/or treatment information from the second user device.

16. The system of claim 1, wherein the sequence of images is encrypted on a first user device, and the encrypted sequence of transformed images are sent to a second user device.

17. The system of claim 15, further comprising a display that displays the diagnosis and treatment information on the device.

18. (canceled)

19. The system of claim 1, where in the system comprises a battery operated handheld mobile device having a processor-implemented module configured to analyze images of skin lesions and determine a progression factor of the skin lesion based on a change in the area of the skin lesions.

20. The system of claim 1, further comprising a light source and a polarizer.

21. (canceled)

22. The system of claim 1, further comprising a light source having a plurality of emitters that emit light at different wavelengths and a detachable light source housing having a battery and a control circuit.

23. (canceled)

24. (canceled)

25. The system of claim 1, wherein the data processor applies a transformation to a plurality of images wherein the transformation comprises a homography transform and the data processor computes an energy minimization function with an iterative computational process.

26. (canceled)

27. (canceled)

28. (canceled)

29. The system of claim 1, wherein the data processor compensates for intensity variation across each image.

30. A computer-implemented method for analyzing a skin lesion in an image, the method comprising:
detecting a first image of the skin lesion;
performing color correction on the first image including performing histogram equalization, with a data processor;
performing contour detection on the first image to identify one or more contours of the skin lesion; and
storing the resulting image with a memory device.

31. The method of claim 30 further comprising receiving a sequence of images wherein the images correspond to the skin lesion represented in the first image, and the sequence of images represent the skin lesion over a period of time.

32. The method of claim 30, further comprising performing color correction on a sequence of images including performing histogram equalization on a color channel or further comprising performing contour detection on the sequence of images to identify one or more contours of the skin lesion or performing feature detection on the sequence of images to identify one or more features of the skin lesion.

33. (canceled)

34. (canceled)

35. The method of claim 30 further comprising storing the resulting sequence of images in a memory device within a handheld camera device.

36. The method of claim 30 further comprising determining a progression factor based on a comparison of an area of the skin lesion in the first image and of the skin lesion in the sequence of images wherein the progression factor is determined based on a comparison of the one or more features of the skin lesion in the first image and the one or more features of the skin lesion in the sequence of images.

37. (canceled)

38. The method of claim 36, wherein the progression factor is determined based on a comparison of the one or more features of the skin lesion in the first image and the one or more features of the skin lesion in the sequence of images.

39. The method of claim 30, wherein the color channel comprises a red color channel, a green color channel, and a blue color channel, and the histogram is performed on each of the color channels independently.

40. The method of claim 30, wherein performing the contour detection comprises performing a level set method using a region-based image segmentation scheme.

41. The method of claim 30, further comprising performing feature detection includes performing a scale invariant feature transform feature matching process.

42. A method for monitoring a progression of a lesion via images, the method comprising:
receiving a sequence of images wherein a first image of the sequence of images is indicated;
performing color correction on the sequence of images including performing histogram equalization on a color channel;
performing contour detection on the sequence of images to identify one or more contours of the skin lesion;
performing feature detection on the sequence of images to identify one or more features of the skin lesion;
storing the resulting sequence of images as transformed images; and
determining a progression factor based on a comparison of an area of the skin lesion in the first transformed image and of the skin lesion in the sequence of transformed images.

43. The method of claim 42, wherein the sequence of images are captured by a user using a mobile device wherein the sequence of images are received at a first user device, and the progression factor is determined at the first user device.

44. (canceled)

45. The method of claim 42, further comprising sending the sequence of transformed images and the progression factor to a second user device;
receiving, at the first user device, diagnosis and treatment information from the second user device and further comprising displaying the diagnosis and treatment information.

46. The method of claim 42, wherein the sequence of images is encrypted on the first user device, and the encrypted sequence of transformed images are sent to the second user device.

47. (canceled)

48. The method of claim 42, wherein the sequence of images are received at a first user device, and the progression factor is determined at a second user device.

49. The method of claim 43, wherein the mobile device comprises a battery operated system including an image detector, a data processor, a display and a wireless transmitter.

50. A system for monitoring a progression of a body region, the system comprising:
a mobile device including an image capturing mechanism; and
the mobile device comprising a processor-implemented module configured to analyze images of a body region and determine a progression factor of the body region based on a change in the condition of the body region.

51. The system of claim 50, further comprising a portable imaging module configured to couple to the image capturing mechanism on the mobile device and to provide lighting to capture images of skin lesions via the image capturing mechanism on the mobile device.

52. The system of claim 50, wherein the processor-implemented module is further configured to perform color correction on the images including performing histogram equalization on a color channel.

53. The system of claim 50 wherein a data processor performs contour detection on the images to identify one or more contours of a skin lesion.

54. The system of claim 50 wherein a data processor performs feature detection on the images to identify one or more features of a skin lesion.

55. The system of claim 50 wherein a data processor determines a progression factor based on a comparison of an area of the skin lesion between the images.

56. The system of claim 50 wherein the color channel comprises a red color channel, a green color channel, and a blue color channel, and the histogram is performed on each of the color channels independently.

57. The system of claim 50, wherein a data processor performs the contour detection comprises performing a level set method using a region-based image segmentation scheme.

58. The system of claim 50, wherein a data processor performs the feature detection includes performing a scale invariant feature transform feature matching.

59. The system of claim 50 wherein the system connects to a detachable light source housing and the housing comprises a polarized.

60. (canceled)

61. The system of claim 50 wherein the mobile device comprises a wireless mobile phone having a virtual keyboard, a battery, a data processor and a light source.

62. (canceled)

63. The system of claim 62 wherein the data processor processes a detected image to compensate for different imaging angles wherein an imaging sensor of the mobile device is oriented along a different alignment axis relative to a body region to be imaged.

64. The system of claim 59 wherein the housing comprises a second battery and a housing controller.

65. The system of claim 59 wherein the housing comprises a plurality of light emitting diodes or the housing comprises a multispectral light source and further comprising a connector to electrically connect the housing to the mobile device.

66. (canceled)

67. (canceled)

68. The system of claim 50 further comprising a graphical user interface operable on a touchscreen display of the mobile device and further comprising a near field communication device within the mobile device to transmit and/or receive data to an external device.

69. (canceled)

70. A non-transitory computer readable medium storing instructions executable by a processing device, wherein execution of the instructions causes the processing device to implement a method for monitoring a progression of a lesion via images comprising:
receiving a sequence of images wherein a first image of the sequence of images is indicated;
performing color correction on the sequence of images including performing histogram equalization on a color channel;
performing contour detection on the sequence of images to identify one or more contours of the skin lesion;
performing feature detection on the sequence of images to identify one or more features of the skin lesion;
storing the resulting sequence of images as transformed images; and
determining a progression factor based on a comparison of an area of the skin lesion in the first transformed image and of the skin lesion in the sequence of transformed images.

71. The non-transitory computer readable medium of claim 70, wherein the sequence of images are captured by a user using a mobile device and wherein the sequence of images are received by a processing device at a first user device, and the progression factor is determined by the processing device at the first user device.

72. (canceled)

73. The non-transitory computer readable medium of claim 70 further comprising:
sending the sequence of transformed images and the progression factor to a processing device at a second user device; and
receiving, at the processing device at the first user device, diagnosis and treatment information from the second user device and wherein the sequence of images is encrypted via the processing device at the first user device, and the encrypted sequence of transformed images are sent to the second user device, and further comprising displaying the stored and treatment information on the device.

74. (canceled)

75. (canceled)

76. The non-transitory computer readable medium of claim 70, wherein the sequence of images are received via a processing device at a first user device, and the progression factor is determined via a processing device at a second user device and further comprising stored instructions to compute an energy minimization function and further comprising polarized image data stored on said medium.

77. (canceled)

78. (canceled)

79. The non-transitory computer readable medium of claim 70 further comprising a level set method and/or a homography transformation.

80. The non-transitory computer readable medium of claim 70 further comprising processing a plurality of images and determining a diagnostic value based upon a plurality of images.
